(19) United States
(12) Patent Application Publication
(10) Pub. No.: US 2014/0074012 A1
(43) Pub. Date: Mar. 13, 2014

(54) COMPOSITION, SYSTEM AND METHOD FOR TISSUE AUGMENTATION

(71) Applicant: Ernesto Andrade, Miami, FL (US)

(72) Inventor: Ernesto Andrade, Miami, FL (US)

(21) Appl. No.: 13/614,601

(22) Filed: Sep. 13, 2012

(51) Int. Cl. C08K 3/10; A61M 37/00; A61M 5/178

(52) U.S. Cl. 604/22; 106/286.7; 604/187; 604/506

(57) ABSTRACT

A composition for purposes of augmentation contains a plurality of substantially spherical, biocompatible semi-solid beads each having a diameter in the range of about 1 millimeter to 10 millimeters. The composition also includes a delivery solvent for sanitary lubrication of the beads. An inventive system and method are utilized to systematically administer the composition into a patient's adipose tissue for customized augmentation. The system can comprise one or more dispensing apparatus, such as in the form of one or more pre-filled syringes. The size and number of pre-filled dispensing apparatus can be selected and organized into one or more kits for use in connection with a specific treatment or group of treatments. The inventive method involves selecting an insertion site in proximity to an identified target tissue, inserting the dispensing apparatus into the tissue through the insertion site, and infiltrating the composition into the tissue in a controlled manner.

### BACKGROUND OF THE INVENTION

1. Field of the Invention

The present invention is directed to a system and method of tissue augmentation which utilizes a safe and unique composition that is systematically administered into a patient's adipose tissue. The inventive composition can be supplied in connection with dispensing apparatus in the form of one or more kits which can be customized for use in connection with a specific treatment or group of treatments.

2. Description of the Related Art

Over the past few decades, the field of cosmetic surgery has transitioned from being a relatively obscure practice, reserved primarily for severely wounded (military, accident victims, etc.) or wealthy consumers, into a more socially acceptable and financially affordable option that is now available to a majority of the population in economically developed countries.

The present day field of cosmetic surgery spans a wide variety of procedures and technologies, targeting different body parts and tissue types, ranging from hair and skin to breasts and buttocks. One particular area of cosmetic surgery commonly referred to as “augmentation” involves adding material (whether natural, synthetic, or a combination thereof) to a desired part of the body to achieve a desired size and/or shape; with breast or buttock enhancements being common examples.

One primary method of accomplishing augmentation involves placement of an implant of predetermined size and shape into a desired treatment area. This is one of the most common augmentation methods, and although highly developed and largely proven, it still entails certain drawbacks. For instance, the precise size and shape of the implant must be determined prior to placement, which leaves little room for adjustment. From a practical standpoint, this limits the potential for fine tuning of the procedure and generally lacks the ability to provide for last-minute modifications to the patient's overall final appearance. Another problem associated with implants is that there is a distinct probability that the patient's body, over time, will ultimately develop an aversion to the implant such that the procedure will eventually need to be repeated with a replacement cosmetic implant. One of the most challenging aspects of repeating such a procedure is that it will likely be substantially invasive such that the patients are sometimes not physically fit for the surgery, or sometimes simply no longer desire to undergo the procedure. It would therefore be advantageous to provide an augmentation system and method which avoids such disadvantages.

Other methods of augmentation involve targeted injection, such as with gels or microspheres, which on the one hand can provide for enhanced customization, while on the other hand present their own disadvantages. For instance, a well known problem with gels and microspheres is that they tend to be absorbed by the patient's body, resulting in potential health implications or, at a minimum, a need for periodically repeated treatment. Moreover, injection of such fluid compositions can lead to problems with migration, clumping, and difficulties in adequately filling the targeted region.

Accordingly, it would be advantageous to provide a highly customizable tissue augmentation system and method that utilizes a composition which enables the primary benefits described above, while avoiding the primary drawbacks. Such a composition, system and method should incorporate safe and stable materials which can be administered in a custom manner. It would be a further advantage for such a system to be available in the form of a kit for ease of use by the practitioner.

### SUMMARY OF THE INVENTION

The present invention is directed to a system and method of tissue augmentation which utilizes a safe and unique composition that is systematically administered into a patient's adipose (or “fat”) tissue to achieve a desired result. The composition can be supplied in connection with dispensing apparatuses, such as pre-filled syringes, in the form of one or more kits. The kits can be customized for use in connection with a specific treatment or group of treatments.

In at least one embodiment, the composition is loaded into a delivery mechanism or dispensing apparatus and pre-packaged for use. The composition primarily comprises a plurality of substantially spherical, biocompatible semi-solid beads each having an outer diameter in the range of about 1 millimeter to about 10 millimeters. Generally, it is preferable from a delivery standpoint that the selected beads for a given volume of composition each comprise a substantially uniform sized outer diameter. In a preferred embodiment, the biocompatible semi-solid beads comprise a medical grade polymer-based material of construction, such as medical grade silicone. Prior to use and/or loading into the dispensing apparatus, the beads are sterilized by a suitable method.

A primary inventive feature of the present invention involves the size of the beads, whose size range was previously thought too large to be practically administered given traditional delivery methods. For instance, it had been generally acknowledged in the art that implant particles of a particular size range greater than just a fraction of a millimeter were simply too large to be injected for use in cosmetic augmentation of the type contemplated herein. However, the present inventive composition and method enables the targeted infiltration of the beads directly into a specific tissue, namely, the adipose tissue to achieve previously unattainable results.

Some of the benefits of utilizing bead sizes disclosed in the present invention include resistance to, or avoidance of, shifting within the tissue; resistance to, or avoidance of, clumping; and resistance to, or avoidance of, absorption by the body. These benefits are not adequately provided by other compositions or methods known in the art. Furthermore, since the inventive composition directly infiltrates the fat tissue, the nature of the adipose tissue itself provides a smoothing effect which provides for greater aesthetic tolerances despite the non-traditional size of the beads.

In at least one embodiment, the composition also includes a delivery solvent structured to provide sanitary lubrication of the beads relative to one another and relative to an interior portion of a delivery mechanism. The delivery solvent can also serve to lubricate the beads upon infiltration into the soft tissue.

The delivery mechanism (dispensing apparatus) can comprise a syringe configuration having an inner chamber structured to releasably contain the composition, and a plunger with an outer end portion that a user presses inward to control dispensing of the composition. Such a dispensing apparatus can further comprise a cannula structured to precisely deliver the composition to the desired location. While the cannula could in some embodiments comprise a simple exit aperture located at a distal end thereof, a preferred embodiment would include at least one transversely oriented aperture.

The various components of the kit assembly include at least one but more practically a plurality of dispensing apparatus, such as syringes, initially disposed in at least one tray. The size and predetermined number of delivery apparatuses can vary depending on the desired treatment. It is further noted that in some embodiments, at least some of the delivery apparatuses will comprise different sizes and/or volumes. For instance, a smaller delivery apparatus may contain a reserve amount of composition, such as for final touches or additional margin of error relative to the anticipated optimum quantity. The tray can include a cover, such as a standard fitting cover, or alternatively, a cover having a thin, sheet structure with securing means. Preferably, the syringes or other suitable dispensing apparatuses are pre-loaded with an appropriate amount of composition such that they can be removed from the tray and used without further preparations.

The kits, like the composition, are assembled in a sterile environment and comprise safe, sterile materials of construction suitable for medical applications. The trays typically comprise a solid, generally rectangular configuration and are ordinarily made of a polyethylene material of construction, such as PETE 1, or similar type of plastic material. Further, the covers can attach to the trays in a variety of ways, such as via a built-in adhesive portion, snap-on fit configuration, separate attachment means, etc.

The method associated with the present invention begins with a surgeon or other practitioner identifying the local tissue to be augmented (e.g., adipose tissue of the chin, cheek, breast, buttocks, etc.). The method further comprises selecting an insertion site and appropriately marking it for entry of the delivery or dispensing apparatus.

Next, a predetermined amount of tumescent fluid or similar type of anesthetic fluid, such as Klein Solution, is injected via traditional means into the target area to prepare the tissue for infiltration of the composition. The tumescent fluid acts as a local anesthetic and also tends to firm up the target tissue. Ordinarily, a predetermined amount of time (e.g., 10 minutes, 15 minutes, etc.) will be allowed to pass subsequent to injecting the tumescent fluid before infiltrating the tissue with the augmentation composition. This provides sufficient time for the anesthetic to take effect.

Once the anesthetic takes effect, a small incision is made into the skin at the insertion site to permit the passage of the delivery apparatus into the adipose tissue.

In at least one embodiment, infiltration of the fat tissue with the composition begins by inserting the tip or distal end of the cannula portion of a pre-filled syringe assembly to a maximum desired depth within the target area of the tissue. The composition is then administered into the adipose tissue by controlled dispensation, wherein the dispensing end of the syringe is simultaneously withdrawn away from the interior distal location of insertion and back towards the insertion site as the composition is dispensed. More specifically, the dispensation of the composition is controlled by depressing the plunger portion of the syringe slowly and evenly at an appropriate rate while the tip of the cannula is slowly withdrawn towards the insertion site, up to within approximately 1-2 inches thereof. This technique can be repeated until a desired quantity of composition is infiltrated into the tissue. This process can also be repeated through various stratifications of tissue to achieve the desired result.

While in some applications a single, pre-loaded syringe will contain a sufficient amount of composition to achieve the desired degree of augmentation, other cases may require use of a plurality of delivery apparatuses, some of which might contain differing amounts of the augmentation composition.

Once the desired result is achieved, the insertion site is closed, such as with an appropriate suture or other surgical closure means. Following closure of the insertion site, the patient should be kept at rest in an appropriate decubitus position for a predetermined amount of time, to allow for initial recovery from the rigors of the procedure.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1A is a schematic side elevation view representation of a portion of one embodiment of a delivery mechanism in accordance with the present invention.

FIG. 1B is a schematic front end view of the embodiment of FIG. 1A.

FIG. 2 is a schematic representation of one embodiment of a system in accordance with the present invention, including the delivery mechanism of FIG. 1A, containing the inventive composition.

FIG. 3A is a schematic perspective view representation of one embodiment of a kit in accordance with the present invention.

FIG. 3B is a schematic plan view representation of another embodiment of a kit in accordance with the present invention.

FIG. 4 is a flow chart representation of a method of the present invention.

FIG. 5 is a schematic representation of a portion of the method of FIG. 4 utilizing the system of FIG. 2.

FIG. 6 is a schematic representation of another portion of the method of FIG. 4 utilizing the system of FIG. 2.

Like reference numerals refer to like parts throughout the several views of the drawings.

### DETAILED DESCRIPTION OF THE PREFERRED EMBODIMENT

As shown in the accompanying Figures, the present invention is directed to a system, generally indicated as 10, and a method, generally indicated as 200, of tissue augmentation which utilizes a safe and unique composition 30 that is systematically administered into a patient's adipose (or “fat”) tissue. The inventive composition 30 can be supplied in connection with dispensing apparatus 20 in the form of one or more kits 100 which can be customized for use in connection with a specific treatment or group of treatments. The inventive tissue augmentation composition 30, system 10 and method 200 provide distinct advantages over the prior art, while avoiding the primary drawbacks.

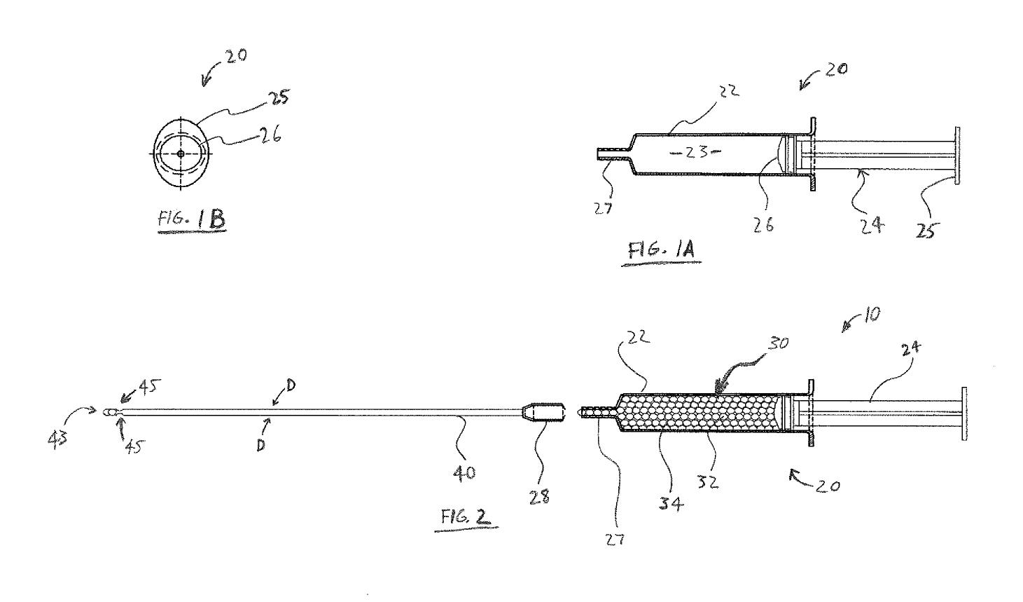

As seen in FIG. 2, the composition 30 is intended to be loaded into a delivery mechanism or dispensing apparatus 20; preferably pre-packaged for use. The composition 30 primarily comprises a plurality of substantially spherical, biocompatible semi-solid beads 32. As used herein, the term “biocompatible” means generally safe for use as an implant material for use in the human body. As used herein, “semi-solid” means generally solid, in contrast to fluid, yet with a rubber-like consistency that generally mimics the feel and density of flesh. In a preferred embodiment, the biocompatible semi-solid beads 32 will comprise a medical grade polymer-based material of construction, such as medical grade silicone. However, other suitable materials with similar properties are also contemplated to be within the scope and intent of the present invention. Prior to use and/or loading into the dispensing apparatus 20, the beads 32 are sterilized by a suitable method. By way of example, suitable sterilization methods can include, but are not limited to, chemical sterilization methods, Ethylene Oxide methods, irradiation methods, etc.

It is emphasized that a primary inventive feature of the present invention involves the size of the beads 32, whose size range was previously thought too large to be practically administered given traditional delivery methods and the pressure differentials associated with the relevant target injection areas. For instance, it had been generally acknowledged in the art that implant particles of a particular size range greater than just a fraction of a millimeter were simply too large to be injected for use in cosmetic augmentation of the type contemplated herein. However, the present inventive composition 30 and method 200 enables the targeted infiltration of biocompatible semi-solid beads 32 each having an outer diameter in the range of about 1 millimeter to about 10 millimeters. More specifically, the method 200, discussed in more detail below, involves infiltrating the beads 32 directly into a specific tissue, namely, the adipose tissue to achieve previously unattainable results. In contrast, previous methods generally focused on injecting implant material into inter-tissue layers, such as the areas between the muscle, skin, and adipose layers, respectively. Accordingly, the present invention utilizes a composition 30 having beads 32 in the aforementioned size range, wherein the more specific size of the beads 32 is selected to generally correspond to appropriate use in a desired target area. Factors in choosing the appropriate bead size can involve size or volume of the target area as well as the desired effect of the infiltration. By way of example, one range of generally categorized bead size has an outer diameter in the range of about 1-2 millimeters; another in the range of about 3-4 millimeters; and another in the range of about 5-10 millimeters. Benefits of utilizing beads 32 of the aforementioned size ranges include resistance to, or avoidance of, shifting within the tissue; resistance to, or avoidance of, clumping; and resistance to, or avoidance of, absorption by the body. These benefits are not adequately provided by other compositions or methods known in the art. Furthermore, since the composition 30 directly infiltrates the fat tissue, the nature of the adipose tissue itself provides a smoothing effect which provides for greater aesthetic tolerances despite the non-traditional size of the beads 32.

In at least one embodiment, the composition 30 also includes a delivery solvent 34 structured to provide sanitary lubrication of the beads 32 relative to one another and relative to an interior portion of a delivery mechanism (dispensing apparatus) 20. The delivery solvent 34 can also serve to lubricate the beads 32 upon infiltration into the soft tissue. As seen in reference to FIGS. 1A and 2, the plurality of beads 32 is immersed in the delivery solvent 34 as the composition 30 is contained within an inner chamber 23 of a dispensing apparatus 20. Preferably, the delivery solvent 34 comprises primarily saline solution, typically 0.9 percent Sodium Chloride (0.9% NaCl) dissolved in water, although a wide variety of other possible concentrations are contemplated to be implemented, as appropriate. It is also within the scope and intent of the present invention to utilize delivery solvents 34 that comprise other suitable chemical compositions. With continued reference to the system 10 depicted in FIGS. 1A, 1B, and 2, the delivery mechanism (dispensing apparatus) 20 can comprise a syringe configuration as shown. In this embodiment, the syringe 20 comprises a body 22 which encloses an inner chamber 23 structured to releasably contain the composition 30. The syringe 20 includes a plunger 24 having an outer end portion 25 that a user presses inward to control dispensing of the composition 30, and an oppositely disposed inner end portion 26 situated on the interior of the chamber 23 which serves to force the composition out of the chamber 23 via the neck 27. The overall shape of the syringe 20 is typical of syringes commonly available in the art. The size of the syringe 20 will depend on the desired volume of composition 30 for a given application. The syringe 20 can comprise a variety of suitable materials readily available to those knowledgeable in the art. By way of example only, one such material can include PETE 1, a medical grade thermoplastic polymer resin often used to construct syringes and other medical apparatus.

The dispensing apparatus 20 further comprises a cannula 40 structured to precisely deliver the plurality of beads 32 to the desired location. As seen in FIG. 2, the cannula 40 comprises an inner diameter D. In at least one embodiment, the inner diameter D of the cannula 40 is selected to accommodate passage of a stream of individual beads 32 having a corresponding bead size. The cannula 40 also preferably comprises a suitable attachment assembly 28 that can facilitate side by side packaging as well as re-use with multiple filled syringe bodies 22. The cannula 40 can thus be connected to the syringe body 22 by connecting the attachment assembly 28 to the corresponding neck portion 27 of the syringe 20. In at least one embodiment, the attachment is achieved by mechanical threading, although a variety of other suitable interconnection means are contemplated as well.

While the cannula 40 of the present invention could in some embodiments comprise a simple exit aperture located at the distal end 43, a preferred embodiment would include at least one transversely oriented aperture 45 for the beads 32 to exit from. As depicted in the embodiment of FIG. 2, the cannula 40 includes two such oppositely disposed, transversely oriented apertures 45. FIG. 2 further depicts a rounded tip 43 which serves to facilitate insertion into the desired target area of adipose tissue. The cannula 40 can comprise any suitable material of construction readily available in the art, including, but not limited to, metals, plastics, composites, etc.

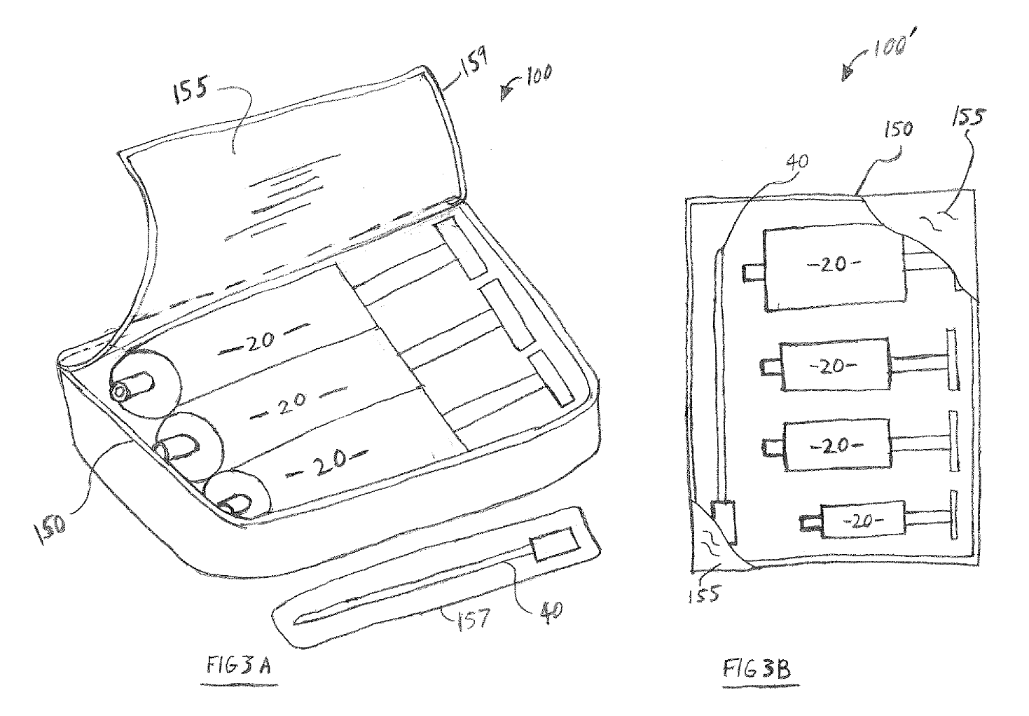

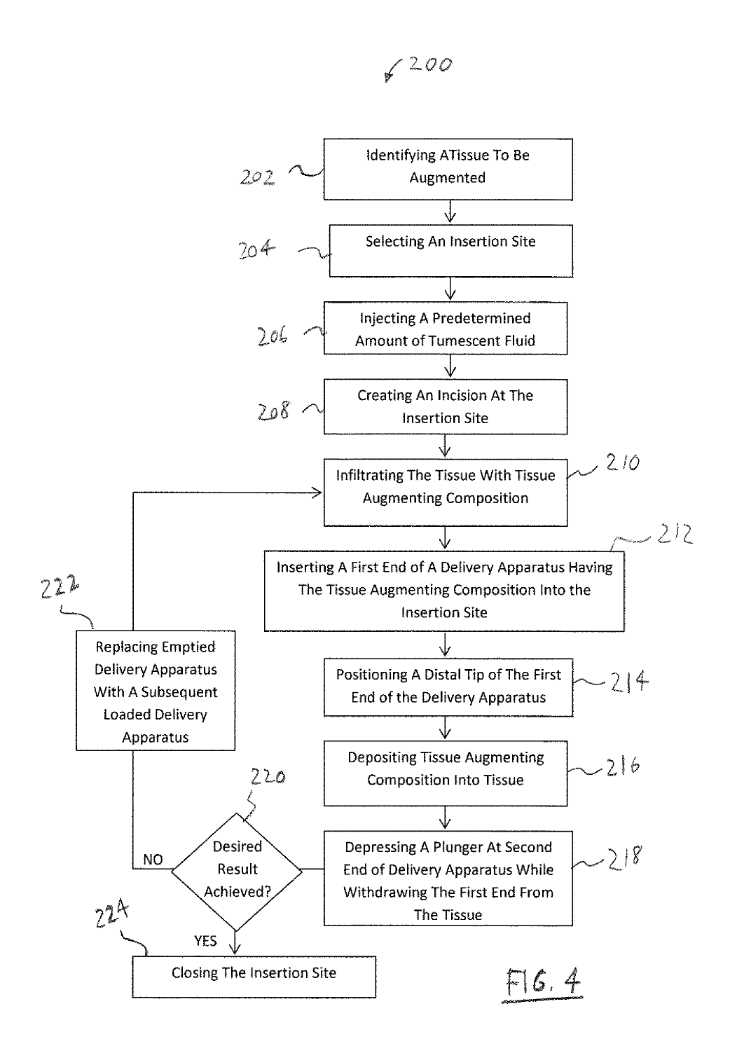

With primary reference to the generic embodiments of FIGS. 3A and 3B, the various components of the kit assembly 100, 100′ are collectively represented and include at least one but more practically a plurality of dispensing apparatus 20 initially disposed in at least one tray 150. The size and predetermined number of delivery apparatus 20 can vary depending on the desired treatment, and as such, the specific kits depicted in FIGS. 3A and 3B are shown for representative purposes and are not to be viewed in a limiting sense. It is further noted that in some embodiments, at least some of the delivery apparatus 20 will comprise the certain size and/or volume, whereas one or more others will comprise a different size and/or volume. For instance, a smaller delivery apparatus 20 may contain a reserve amount of composition 30, such as for final touches or additional margin of error relative to the anticipated optimum quantity.

The tray 150 can include a cover 155, such as a standard fitting cover, or alternatively, a cover having a thin, sheet structure with securing means. The kit 100 can also include one or more cannulas 40 which connect to the rest of the syringe or other suitable dispensing apparatus 20. In some embodiments, the cannulas 40 are provided outside of the tray 150 and may include separate packaging 157. In other embodiments, the cannulas 40 are included with the rest of the dispensing apparatus 20 within the trays 150. Preferably, the syringes 20 are pre-loaded with an appropriate amount of composition 30 such that they can be removed from the tray 150 and used without further preparations. As noted, above, the size and number of the syringes 20, and also the size of the beads 32, can be selected based on a desired type and/or location of treatment.

By way of example, in one embodiment, the kit 100 can comprise one 2.0 milliliter syringe 20 pre-filled with a composition 30 that includes a plurality of 2 millimeter diameter medical grade silicone spheres 32 immersed in a saline solution 34. The respective parameters of such a kit may correspond to a specific desired type of treatment such as, in this example, a smaller type of body area which may include the chin and/or neck area. In other cases, a kit 100 for augmentation of a larger body area, such as the gluteal area, can include a plurality of larger syringes 20. One specific example includes ten 20.0 milliliter syringes 20, each pre-filled with a composition 30 of 3 or 4 millimeter diameter semi-solid spheres 32 immersed in delivery solvent 34. Generally, it is preferable from a delivery standpoint that the selected beads 32 for a given volume of composition 30 each comprise a substantially uniform sized outer diameter.

The kits 100, like the composition 30, are assembled in a sterile environment and comprise safe, sterile materials of construction suitable for medical applications. In particular, the trays 150 can comprise a variety of shapes, configurations, and materials. As seen in FIGS. 3A-3B, the trays 150 typically comprise a solid, generally rectangular configuration and are ordinarily made of a polyethylene material of construction, such as PETE 1, or similar type of plastic material. The corresponding covers 155, as well as the optional separate packaging 157, can also be made of plastic or other suitable material, such as polyethylene spun fiber or TYVEK sheets. Further, the covers 155 can attach to the trays 150 in a variety of ways, such as via a built-in adhesive portion 159, snap-on fit configuration, separate attachment means, etc.

The various components of the kits 100 can be sterilized by variety of methods including, but not limited to, chemical sterilization methods, Ethylene Oxide methods, irradiation methods, etc. In at least one preferred embodiment, the assembled kits 100 are sterilized utilizing a STERRAD radiation sterilization system.

With primary reference now to FIGS. 4-6, the present invention contemplates associated methods 200, utilizing the above-described composition 30, system 10 and/or kits 100.

Turning first to FIG. 4, the method 200 associated with the present invention begins at 202, wherein a surgeon or other practitioner identifies the local tissue to be augmented (e.g., adipose tissue of the chin, cheek, breast, buttocks, etc.). As seen throughout the Figures, the adipose tissue “A” generally lies beneath the skin tissue “S.”

As illustrated at 204, the method 200 further comprises selecting an insertion site “X” and appropriately marking it for entry of the delivery or dispensing apparatus 20. Directional indication markers, such as those indicating one or more directional axes, may also be utilized to facilitate the administration of the composition 30 by the delivery apparatus 20.

Next, a predetermined amount of tumescent fluid or similar type of anesthetic fluid, such as Klein Solution, is injected via traditional means into the target area to prepare the tissue for infiltration of the composition 30, as at 206. The tumescent fluid acts as a local anesthetic and also tends to firm up the target tissue. The tumescent fluid can comprise a predetermined amount of anesthetic (such as Xylocaine, e.g. 0.1% solution) as well as predetermined amounts of other optional substances, such as vasoconstrictor (e.g. Epinephrine), Sodium Bicarbonate, etc. The procedure 200 may also be performed by administering other types of general, regional, or local anesthesia to the patient. Ordinarily, a predetermined amount of time (e.g., 10 minutes, 15 minutes, etc.) will be allowed to pass subsequent to injecting the tumescent fluid before infiltrating the tissue with the augmentation composition 30. This provides sufficient time for the anesthetic to take effect.

Once the anesthetic takes effect, a small incision is made into the skin S at the insertion site X, as at 208. The incision permits the passage of the delivery apparatus 20 into the adipose tissue A, for infiltrating the tissue A with the augmenting composition 30, which is generally indicated at 210. By way of example, the incision can be made with a #15 scalpel or similar-type surgical tool to provide sufficient clearance for inserting an appropriate portion of the delivery apparatus 20 (such as a cannula 40) while minimizing the subsequent effort needed to close the incision upon completion of the procedure.

In at least one embodiment, infiltration of the fat tissue A with the composition 30, as at 210, is accomplished by the specific technique illustrated at 212-218 of FIG. 4. More specifically, the method 200 continues at 212 with insertion of the delivery apparatus 20, such as initially the tip or distal end 43 of the cannula 40 portion of a syringe assembly 20. The syringe assembly 20 is preferably pre-filled with an appropriate quantity of composition 30, as discussed above.

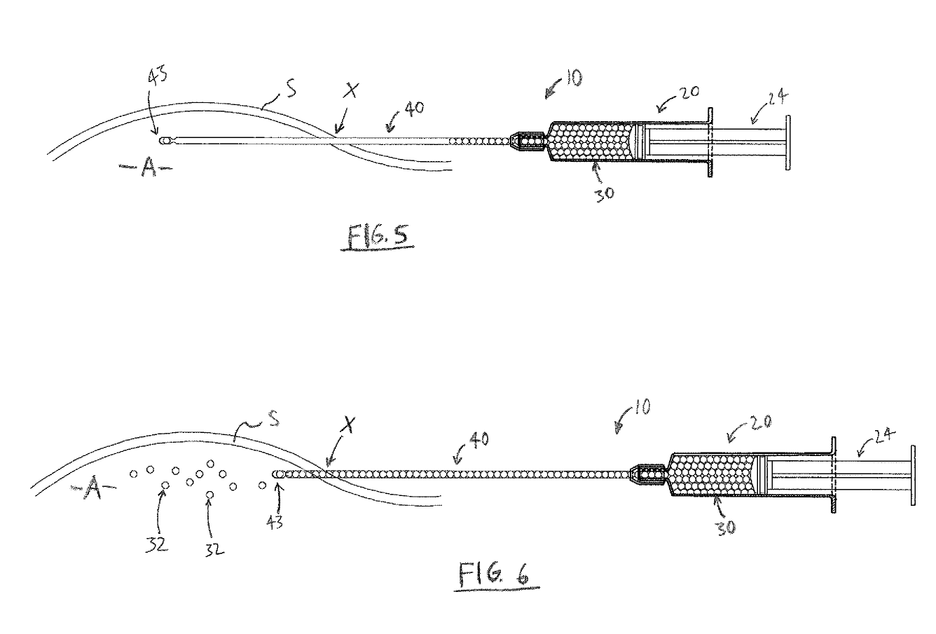

Next, as also seen in FIG. 5, the tip 43 of the cannula 40 is positioned to a maximum desired depth within the target area of the tissue A, as at 214. The composition 30 is then administered into the adipose tissue A, as generally indicated at 216. As also shown in FIG. 6, the depositing of the composition 30 into the tissue A is preferably achieved by controlled dispensation, as at 218 of FIG. 4, wherein the dispensing end 43 of the delivery apparatus 20 is simultaneously withdrawn away from the interior distal location of insertion and back towards the insertion site X as the composition 30 is dispensed. In at least one embodiment, the dispensation of the composition 30 is controlled by depressing the plunger 24 portion of a syringe 20 slowly and evenly at an appropriate rate while the tip 43 of the cannula 40 is slowly withdrawn towards the insertion site X, up to within approximately 1-2 inches thereof. This technique can be repeated until a desired quantity of composition 30 is infiltrated into the tissue A. In this way, the composition 30 is distributed to achieve the desired effect. This process can also be repeated through various stratifications of tissue A to achieve the desired result.

While in some applications a single, pre-loaded syringe 20 will contain a sufficient amount of composition 30 to achieve the desired degree of augmentation, other cases may require use of a plurality of delivery apparatuses 20, as at 220, perhaps of varied volumes and/or containing varied amounts of the augmentation composition 30, as described above. In those cases, the technique is repeated after replacing the emptied delivery apparatus 20 with one or more subsequent loaded delivery apparatus 20, as at 222. In cases where a kit 100 is utilized, if the procedure 200 is completed without using all of a plurality of delivery apparatuses 20, then the tray 150 can be sealed using the appropriate cover 155 for later use. The kit 100 can also be re-sterilized accordingly.

Once the desired result is achieved, as at 220, the insertion site X is closed, as at 224, such as with an appropriate suture or other surgical closure means. For instance, as one example, a Polypropylene suture, (e.g., PROLENE 5-0 suture) can be used. Following closure of the insertion site X, the patient should be kept at rest in an appropriate decubitus position for a predetermined amount of time, to allow for initial recovery from the rigors of the procedure 200.

It should also be noted that although the present invention is primarily intended for use on humans, it could be applied to other beings, such as to dogs, horses, etc., should such a need arise.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

1. A composition for infiltrating soft tissue for purposes of augmentation, comprising:
a plurality of substantially spherical, biocompatible semi-solid beads each having an outer diameter of at least 5 millimeters, and
a lubricative delivery solvent structured to provide sanitary lubrication of said beads relative to one another and relative to an interior of a delivery mechanism and to further lubricate said beads upon infiltration into the soft tissue, wherein said plurality of beads comprise a substantial majority of said composition such that said delivery solvent and said beads do not collectively form a suspension.

2. (canceled)

3. (canceled)

4. A composition as recited in claim 1 wherein said beads comprise an outer diameter in the range of 5-10 millimeters.

5. A composition as recited in claim 1 wherein said beads comprise a medical grade silicone material of construction.

6. A composition as recited in claim 1 wherein said delivery solvent comprises a saline solution.

7. A composition as recited in claim 6 wherein said saline solution comprises about 0.9 percent sodium chloride dissolved in water.

8. A composition as recited in claim 1 wherein a predetermined quantity of said beads each comprise a substantially uniform sized outer diameter.

9. A kit for infiltrating soft tissue for purposes of augmentation, comprising:
a predetermined number of dispensing apparatus, said dispensing apparatus each initially containing a predetermined quantity of a composition;
said composition comprising a plurality of substantially spherical, biocompatible semi-solid beads each having a diameter in a range of about 1 millimeter to 10 millimeters, and a delivery solvent structured to provide sanitary lubrication of said beads relative to one another and relative to an interior of said dispensing apparatus and to further lubricate said beads upon infiltration into the soft tissue;
wherein said predetermined number of dispensing apparatus and said predetermined quantity of said composition contained in each respective dispensing apparatus is selected to correspond to a predetermined treatment.

10. A kit as recited in claim 9 comprising at least two of said dispensing apparatus, at least one of which contains a different amount of said composition than at least one other of said dispensing apparatus.

11. A kit as recited in claim 9 wherein said beads comprise an outer diameter in the range of about 1-2 millimeters.

12. A kit as recited in claim 11 comprising one dispensing apparatus initially containing about 2.0 milliliters of said composition.

13. A kit as recited in claim 9 wherein said beads comprise an outer diameter in the range of about 3-4 millimeters.

14. A kit as recited in claim 13 comprising ten dispensing apparatus each initially containing about 20.0 milliliters of said composition.

15. A kit as recited in claim 9 wherein said beads comprise an outer diameter in the range of about 5-10 millimeters.

16. A kit as recited in claim 9 wherein said beads comprise a medical grade silicone material of construction.

17. A kit as recited in claim 9 wherein said delivery solvent comprises a saline solution.

18. A kit as recited in claim 9 wherein said beads contained in each respective dispensing apparatus comprise a substantially uniform sized outer diameter.

19. A method of tissue augmentation comprising:
identifying at least one tissue to be augmented,
selecting an insertion site in proximity to the identified tissue to be augmented, and
infiltrating a tissue augmenting composition into the identified tissue through the insertion site, the tissue augmenting composition including a delivery solvent and a plurality of biocompatible, semi-solid beads each having a diameter within a range of about 1 millimeter to 10 millimeters.

20. The method as recited in claim 19 wherein the semi-solid beads each comprise a substantially spherical shape.

21. The method as recited in claim 19 wherein the semi-solid beads each comprise a medical grade silicone material of construction.

22. The method as recited in claim 19 wherein the at least one tissue to be augmented is adipose tissue.

23. The method as recited in claim 19 further comprising repeatedly infiltrating the tissue augmentation composition into the identified tissue until a desired aesthetic result is achieved.

24. The method as recited in claim 19 wherein the tissue augmenting composition is initially carried in at least one delivery apparatus, and infiltration occurs by administering the tissue augmenting composition from the at least one delivery apparatus into the identified tissue.

25. The method as recited in claim 24 further comprising infiltrating the tissue augmentation composition into the identified tissue by dispending from sequential different ones of a plurality of delivery apparatuses, each including a predetermined amount of tissue augmenting composition, until a desired aesthetic result is achieved.

26. The method as recited in claim 19 further comprising injecting a predetermined amount of tumescent fluid into the section of tissue to be augmented to prepare the tissue for augmentation.

27. A method of tissue augmentation comprising:
identifying an adipose tissue to be augmented,
selecting an insertion site in proximity to the identified adipose tissue to be augmented,
creating an incision at the insertion site,
inserting a first end of a delivery apparatus containing a tissue augmenting composition including a delivery solvent and a plurality of substantially spherical, silicone beads, each having a diameter within a range of about 1 millimeter to 10 millimeters, into the identified tissue through the incision at the insertion site,
positioning the first end of the delivery apparatus to a maximum desired depth in the identified adipose tissue, and
dispensing the tissue augmenting composition into the tissue in a controlled manner while simultaneously withdrawing the first end of the delivery apparatus from the tissue so as to deposit the tissue augmenting composition to achieve a desired effect.

28. The method as recited in claim 27 wherein the delivery apparatus comprises at least one syringe.

29. The method as recited in claim 28 wherein the first end of the delivery apparatus comprises a tip of a cannula.

30. The method as recited in claim 29 wherein dispensing the tissue augmenting composition into the tissue in a controlled manner comprises depressing a plunger associated with a second end of the delivery apparatus.

31. The method as recited in claim 27 wherein the beads comprise an outer diameter in the range of about 1-2 millimeters.

32. The method as recited in claim 27 wherein the beads comprise an outer diameter in the range of about 3-4 millimeters.

33. The method as recited in claim 27 wherein the beads comprise an outer diameter in the range of about 5-10 millimeters.

34. The method as recited in claim 27 further comprising injecting a predetermined amount of tumescent fluid into the section of tissue to be augmented to prepare the tissue for augmentation.

35. The method as recited in claim 34 wherein the tumescent fluid includes a predetermined amount of vasoconstrictor.

36. The method as recited in claim 34 further comprising allowing a predetermined amount of time to pass after injecting the tumescent fluid before infiltrating with the tissue augmentation composition.
